(19) United States
(12) Patent Application Publication
(10) Pub. No.: US 2014/0309553 A1
(43) Pub. Date: Oct. 16, 2014

(54) KITS AND METHODS FOR SEPARATING A TARGET ANALYTE FROM A SUSPENSION

(71) Applicant: RareCyte, Inc., Seattle, WA (US)

(72) Inventors: Paul Spatafore, Bothell, WA (US); Steve Quarre, Woodinville, WA (US); Jonathan Lundt, Seattle, WA (US)

(73) Assignee: RareCyte, Inc., Seattle, WA (US)

(21) Appl. No.: 14/248,890

(22) Filed: Apr. 9, 2014

Related U.S. Application Data
(60) Provisional application No. 61/810,824, filed on Apr. 11, 2013.

(51) Int. Cl. G01N 1/40; A61B 5/15; A61B 5/153

(52) U.S. Cl. CPC G01N 1/405; A61B 5/153; A61B 5/150221; A61B 5/150229
USPC 600/579; 600/576

(57) ABSTRACT

This disclosure is directed to kits and methods for extracting a target analyte from a biological fluid in real time. The biological fluid, therefore, does not need to be stored or shipped/delivered. Isolating the target analyte in real-time allows for on-the-spot processing with minimal, if any, down time between collection and testing. Furthermore, isolating in real time may also allow for the biological fluid to be extracted, processed, and returned—similar to that of dialysis—to permit for more volume to be processed and tested. In one aspect, an inner surface of a conduit may include a coating having a high affinity for the target analyte. The conduit may also include features which create turbulent flow to permit all portions of the biological fluid to come into contact with the coating. In another aspect, a capture chip is used to isolate the target analyte from the biological fluid.

### CROSS-REFERENCE TO A RELATED APPLICATION

This application claims the benefit of Provisional Application No. 61/810,824, filed Apr. 11, 2013.

### TECHNICAL FIELD

This disclosure relates generally to fluid separation and, in particular, to separating a target analyte from a suspension by passing the suspension through a conduit.

### BACKGROUND

Suspensions often include materials of interest that are difficult to detect, extract and isolate for analysis. For instance, whole blood is a suspension of materials in a fluid. The materials include billions of red and white blood cells and platelets in a proteinaceous fluid called plasma. Whole blood is routinely examined for the presence of abnormal organisms or cells, such as ova, fetal cells, endothelial cells, parasites, bacteria, and inflammatory cells, and viruses, including HIV, cytomegalovirus, hepatitis C virus, and Epstein-Barr virus. Currently, practitioners, researchers, and those working with blood samples try to separate, isolate, and extract certain components of a peripheral blood sample for examination. Typical techniques used to analyze a blood sample include the steps of smearing a film of blood on a slide and staining the film in a way that enables certain components to be examined by bright field microscopy.

On the other hand, materials of interest composed of particles that occur in very low numbers are especially difficult if not impossible to detect and analyze using many existing techniques. Consider, for instance, circulating tumor cells (“CTCs”), which are cancer cells that have detached from a tumor, circulate in the bloodstream, and may be regarded as seeds for subsequent growth of additional tumors (i.e., metastasis) in different tissues. The ability to accurately detect and analyze CTCs is of particular interest to oncologists and cancer researchers, but CTCs occur in very low numbers in peripheral whole blood samples. For instance, a 7.5 ml sample of peripheral whole blood that contains as few as 5 CTCs is considered clinically relevant in the diagnosis and treatment of a cancer patient. However, detecting even 1 CTC in a 7.5 ml blood sample may be clinically relevant and is equivalent to detecting 1 CTC in a background of about 50 billion red and white blood cells. Using existing techniques to find, isolate and extract as few as 5 CTCs of a whole blood sample is extremely time consuming, costly and may be impossible to accomplish.

As a result, practitioners, researchers, and those working with suspensions continue to seek systems and methods to more efficiently and accurately detect, isolate and extract target materials of a suspension.

### DESCRIPTION OF THE DRAWINGS

FIG. 1 shows an exploded view of an example kit.

FIG. 2A shows an exploded view of an example kit.

FIG. 2B shows an example capture chip.

FIG. 3A shows a cross-section of an example conduit.

FIG. 3B shows fluid flow through the example conduit.

FIG. 4 shows a cross-section of an example conduit.

### DETAILED DESCRIPTION

This disclosure is directed to kits and methods for extracting a target analyte from a biological fluid in real time. The biological fluid, therefore, does not need to be stored or shipped/delivered. Isolating the target analyte in real-time allows for on-the-spot processing with minimal, if any, down time between collection and testing. Furthermore, isolating in real time may also allow for the biological fluid to be extracted, processed, and returned—in a manner similar to that of dialysis—to permit for more volume to be processed and tested. In one aspect, an inner surface of a conduit may include a coating having a high affinity for the target analyte. The conduit may also include features which create turbulent flow to permit all portions of the biological fluid to come into contact with the coating. In another aspect, a capture chip is used to isolate the target analyte from the biological fluid.

The detailed description is organized into two subsections: (1) A general description of conduit and puncture system is provided in a first subsection; and (2) using the conduit to separate a target analyte from a suspension is provided in a second subsection.

General Description of Conduit and Venipuncture Systems

A conduit, such as tubing, may be used in a venipuncture kit or in a withdraw-return loop, in which the blood is withdrawn, processed, and returned to the body. The venipuncture approach permits a set amount of a suspension, such as blood, to be withdrawn without having to return the blood. The withdraw-return loop permits a greater amount of blood to be withdrawn, since it is eventually returned to the body, such that the body is not depleted of a large volume of blood at any one given point in time. For the sake of convenience, the methods are described with reference to an example suspension of anticoagulated whole blood. But the methods described below are not intended to be so limited in their scope of application. The methods, in practice, can be used with any kind of suspension and are not intended to be limited to immunotherapeutic analytes designed to interact with components found only in whole blood. For example, a sample suspension can be urine, blood, bone marrow, cystic fluid, ascites fluid, cerebrospinal fluid, nipple aspirate fluid, saliva, amniotic fluid, vaginal secretions, mucus membrane secretions, aqueous humor, vitreous humor, and any other physiological fluid. It should also be understood that a target analyte can be a cell, such as ova or a circulating tumor cell (“CTC”), a circulating endothelial cell, a vesicle, a liposome, a protein, a nucleic acid, a biological molecule, a naturally occurring or artificially prepared microscopic unit having an enclosed membrane, parasites, microorganisms, viruses, or inflammatory cells.

Venipuncture is a process by which blood is collected from a patient or test subject by puncturing a blood vessel or port with a needle or the like. A venipuncture kit typically includes a first needle, a conduit, and a vessel. The first needle can be connected to the vessel, such as a tube, bag, or the like, via the conduit. The conduit may be directly connected to the first needle (i.e. the first needle and tube are one piece) or indirectly connected to the first needle (i.e. a female end of the first needle accepts a male end of the conduit which interlock via complementary threads, clips, or the like). The venipuncture kit may also include an adapter. The adapter includes a cavity and a male or female end to connect to a male or female end of the conduit. The cavity of the adapter may further include a second needle to puncture a cap of the tube, thereby allowing the collected blood to freely flow into the tube. When the conduit is used in a withdraw-return loop, a machine may be required to continuously withdraw and return the blood, though other appropriate methods may be used. When a machine is used, the target analyte can still be extracted within the conduit, as the machine is used to aid in continuously withdrawing and returning the blood.

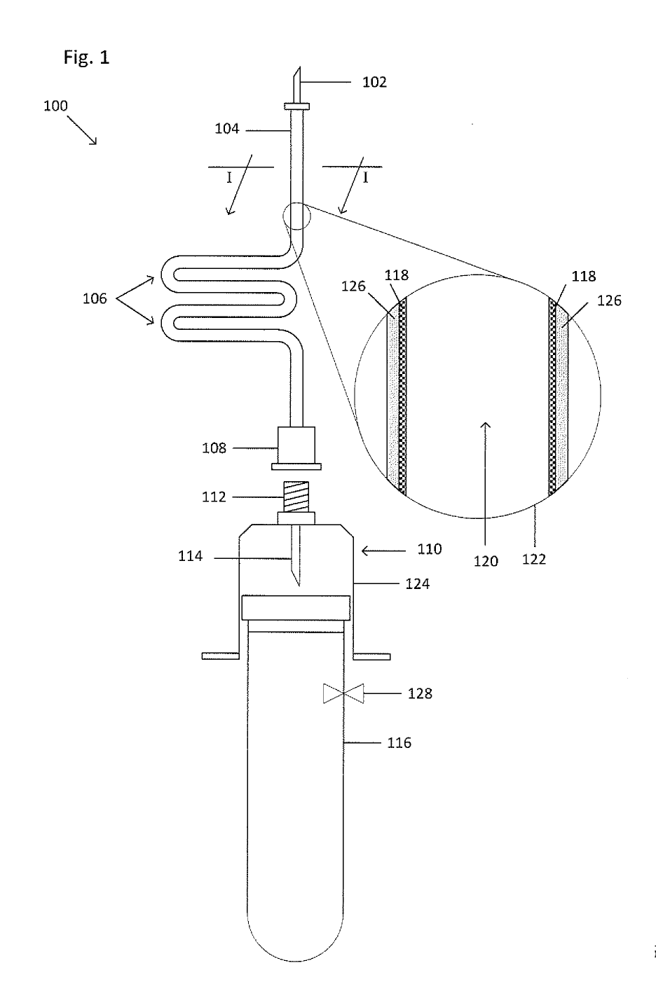

FIG. 1 shows an exploded view of an example kit 100. The kit 100 includes a conduit 104 having a first end and a second end, a needle 102 at the first end of the conduit 104, and a collection vessel 116. The kit 100 may also include an adapter 110 and a connector 108 at the second end of the conduit 104. The adapter 110 may include a body portion 124, a second needle 114 to puncture a cap or top of the collection vessel 116, and a complementary connector 112 to connect to the connector 108 at the second end of the conduit 104. Alternatively, the conduit 104 may include a puncture needle (not shown) at the second end to puncture a cap or top of the collection vessel 116, thereby directly linking to the collection vessel 116 or to return the biological fluid to the patient in a withdraw-return loop. The conduit 104 may chicanes 106, so as to increase the inner surface area exposed to the blood while reducing or maintaining the distance that the conduit 104 extends. The conduit 104 includes an inner channel 120 to permit flow of the blood through the conduit 104, and a wall 126 that encompasses the inner channel 120 to confine the blood within the conduit 104, the wall 126 including an inner surface and an outer surface. The conduit 104 may also include a flow control (not shown) to regulate flow of the biological fluid through the conduit 104 and into the collection vessel 116. The conduit 104 may also include a pressure control (not shown) to regulate the pressure differential between the collection vessel 116 and the needle 102.

To capture a target analyte in real time, an inner surface of the conduit 104 includes a coating 118. The coating 118 increases the affinity or adhesion of the conduit 104 for the target analyte. By increasing the affinity through mechanisms such as adhesion or chemical attraction or bonding, the conduit 104 may capture the target analyte. The enhanced holding of the target analytes to the conduit 104 may also decrease or eliminate the risk of any target analytes being washed away during at least one flow-thru of a fixative, permeabilizing agent, and/or label. The coating 118, located on an inner surface of the conduit 104, may cover a portion of the inner surface, many portions of the inner surface, or the entire inner surface. The coating 118 may form a chemical bond with the target analyte, the bond, and related attraction, may be covalent, ionic, dipole-dipole interactions, London dispersion forces, van der Waal's forces, hydrogen bonding, or any appropriate chemical bond. The needle 102 may also include the coating 118 on an inner portion, an outer portion, or both the inner and outer portions.

The collection vessel 116 may include a pressure regulator 128 to reduce a pressure differential between the collection vessel 116 and the blood vessel (not shown) of the subject. By reducing the pressure differential, the blood may flow through the kit 100 and enter the collection vessel 116 more gently. The pressure regulator 128 may be a valve, a vacuum pump, or the like. The pressure regulator 128 may located in a wall of the collection vessel 116, a cap of the collection vessel 116, or any appropriate place on or within the collection vessel 116. Alternatively, the pressure regulator 128 may be connected to the collection vessel 116 by a port (not shown) within the collection vessel 116. The pressure regulator 128 may also be configured to maintain a constant pressure within the collection vessel 116, thereby maintaining a constant pressure differential between the collection vessel 116 and the blood vessel (not shown), once an appropriate pressure differential has been obtained. The pressure regulator 128 may be fixed or automatically controlled, such as by a sensor. Alternatively, a flow regulator may be used.

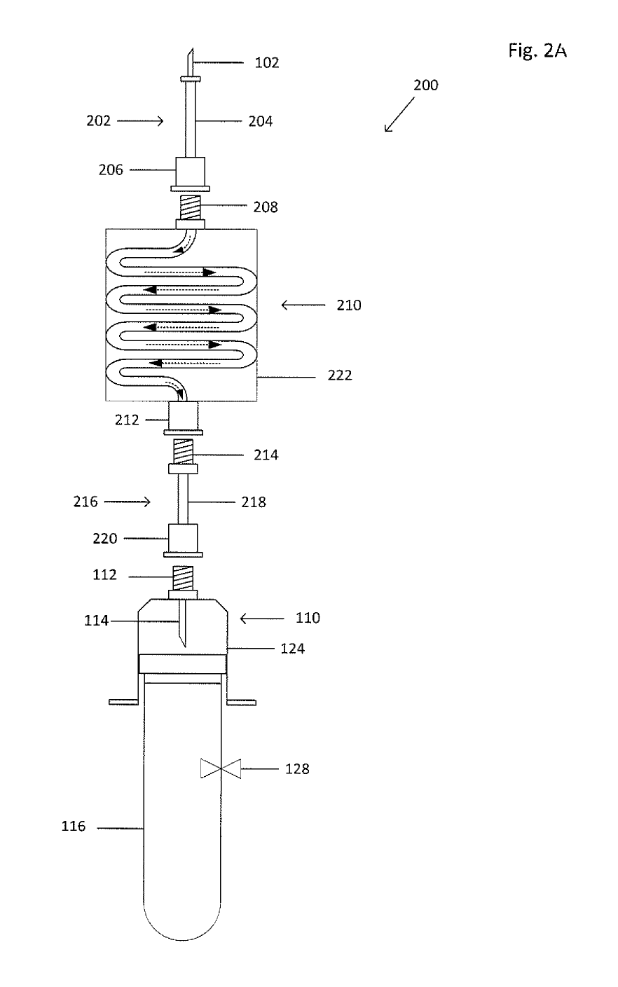

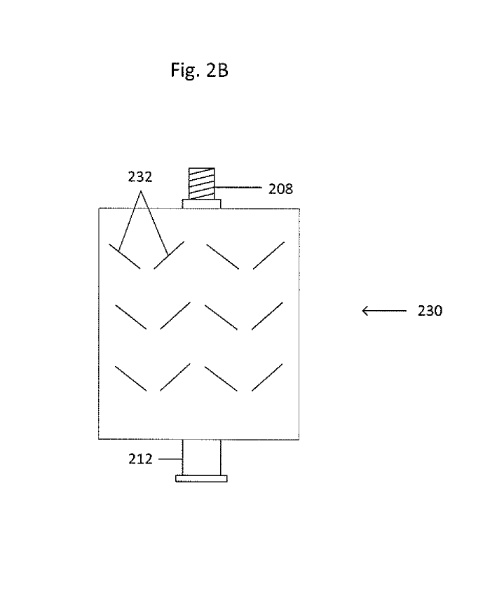

FIG. 2A shows an exploded view of an example kit 200. The kit 200 includes a first conduit segment 202, a capture chip 210, a second conduit segment 216, and a collection vessel 116. The first conduit segment includes a first needle at a first end and a first connector 206 at a second end. The second conduit segment includes a second connector 214 at a first end. The capture chip 210 includes an inlet 208 to connect to the first connector 206 of the first conduit segment 206, a capture body 222, and an outlet 212 to connect to the second connector 214 of the second conduit segment 216. The kit 200 may also include an adapter 110 and a third connector 220 at the second end of the second conduit segment 216. The adapter 110 may include a body portion 124, a second needle 114 to puncture a cap or top of the collection vessel 116, and a complementary third connector 112 to connect to the third connector 220. Alternatively, the second end of the second conduit segment 216 may include a puncture needle (not shown) to puncture a cap or top of the collection vessel 116, thereby directly linking to the collection vessel 116 or to return the biological fluid to the patient in a withdraw-return loop.

The capture chip 210 is separable from the conduit segments 202 and 216. The capture chip 210 may be individually processed and/or used to store the target analyte extracted from the blood that has flowed through. The capture body The chip protrusion 224 may include a single flow channel, as seen in FIG. 2A. The single flow channel may include at least one chicane. FIG. 2B shows an example capture chip 23. The capture chip 230 may include a chip protrusions 232 with a coating having a high affinity for the target analyte. The chip protrusions 232 may be a discontinuous chevron, a pair of parallel lines, bumps, columns, or the like. The chip protrusion 224 may aid in directing fluid flow from the inlet to the outlet.

The collection vessel 116 may include a pressure regulator 128 to reduce a pressure differential between the collection vessel 116 and the blood vessel (not shown) of the subject. By reducing the pressure differential, the blood may flow through the kit 100 and enter the collection vessel 116 more gently. The pressure regulator 128 may be a valve, a vacuum pump, or the like. The pressure regulator 128 may located in a wall of the collection vessel 116, a cap of the collection vessel 116, or any appropriate place on or within the collection vessel 116. The pressure regulator 128 may also be configured to maintain a constant pressure within the collection vessel 116, thereby maintaining a constant pressure differential between the collection vessel 116 and the blood vessel (not shown), once an appropriate pressure differential has been obtained.

The coating may include a capture molecule such as a primary antibody that binds to biomarkers, including but not limited to, EpCAM, AMACR, Androgen receptor, CD146, CD227, CD235, CD24, CD30, CD44, CD45, CD56, CD71, CD324, CD325, MUC1, CEA, cMET, EGFR, Folate receptor, HER2, Mammaglobin, or PSMA.

The coating may also be functionalized to attract or attach target analytes to the conduit using a self-assembled monolayer comprising a head, a tail, and a functional group. The head reacts with and attaches to the surface, and may be any chemical having a high affinity for the surface. The tail can be a carbon backbone that connects the head to the functional group and may be any suitable length and may or may not be branched. The functional group is selected based on the appropriate functionality or reaction desired. After the coating has been functionalized, materials may be added to provide better capture of the target analytes. The materials include Mytilus edulis foot protein (“Mefp”); biopolymers; polyphenolic proteins (including those polyphenolic proteins containing L-DOPA); chemo-attractant molecules, such as epidermal growth factor (“EGF”) or vascular endothelial growth factor (“VEGF”); an extracellular matrix protein (“ECM”); maleic anhydride; maleimide activated sulfa-hydryl groups, poly-L-lysine; poly-D-lysine; streptavidin; neutravidin; protein A; protein G; protein A/G, protein L; biotin; glutathione; antibodies; recombinant antibodies; aptamers; RGD-peptides; fibronectin; collagen; elastin; fibrillin; laminin; or proteoglycans.

The coating may also include a chemo-attractant molecule. Chemo-attractant molecules are ones which will elicit a chemotaxis response from the target analyte, whereby the target analyte is attracted to the chemicals. Chemotaxis is an active movement of the target analyte due to a chemical or chemicals present in the environment. The EGF, VEGF, chemo-attractant molecule, or ECM may be used as a layer, either alone or layered in conjunction with a material discussed above. Furthermore, the EGF, VEGF, chemo-attractant molecule, or ECM may be mixed together as one layer on the outer surface of the conduit. The EGF, VEGF, chemo-attractant molecule, or ECM, when used in combination with one of the other coatings discussed above, may be a sub-layer in which it is layered between the conduit and the other coating or may be the coating where the one or the other materials discussed above is the sub-layer. The coating may also be a mixture of the EGF, VEGF, chemo-attractant molecule, or ECM with one of the materials discussed above. The coating of EGF, VEGF, chemo-attractant molecule, or ECM can cause the target analyte to migrate towards the conduit the surface, where the target analyte can then be captured and held by one of the other coatings discussed above. When the coating of EGF, VEGF, chemo-attractant molecule, or ECM is used separately, it will be the only coating and will simply be more attractive to the target analyte than other surfaces within the tube and conduit system.

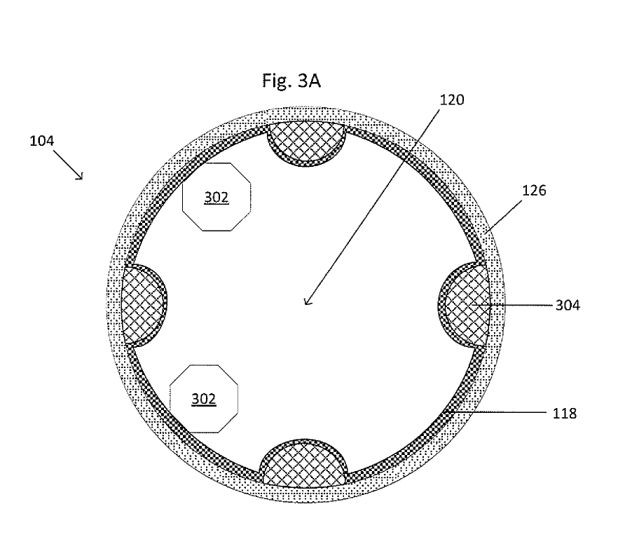

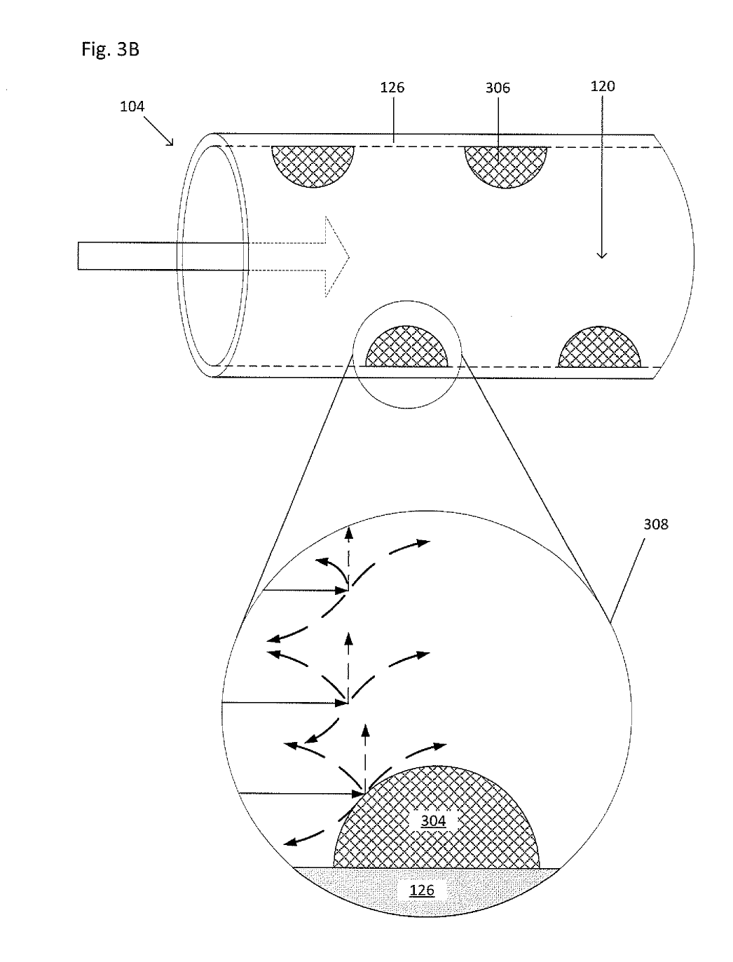

FIG. 3A shows a cross-section of the conduit 104 take along a line I-I. The inner surface of the conduit 104 may also include protrusions 304 to create turbulent flow within the conduit 104, as seen in FIG. 3B. It should be understood that “turbulent flow” means any type of flow that is non-laminar and may therefore include eddies, vortices, or the like. The protrusions 304 permit all portions of the blood to come into contact with the coating 118 by creating turbulent flow. When all of the portions of the blood contact the coating 118, all of the target analytes contact the coating and are therefore capable of being captured by the coating. The protrusions 304 may extend from the inner surface towards the center of the conduit 104. The protrusions 304 may bisect the inner channel 120, may extend halfway through the inner channel 120, or may extend any distance into the inner channel 120. The protrusions 304 may include, but are not limited to, a helical ridge, a bump, a ridge extending circumferentially around the inner surface, or a post. The post may be any shape, including, but limited to, cylindrical, triangular, quadrilateral, polyhedron, conical, frustoconical, spherical, or the like. Additionally, the protrusions 304 may include the coating 118.

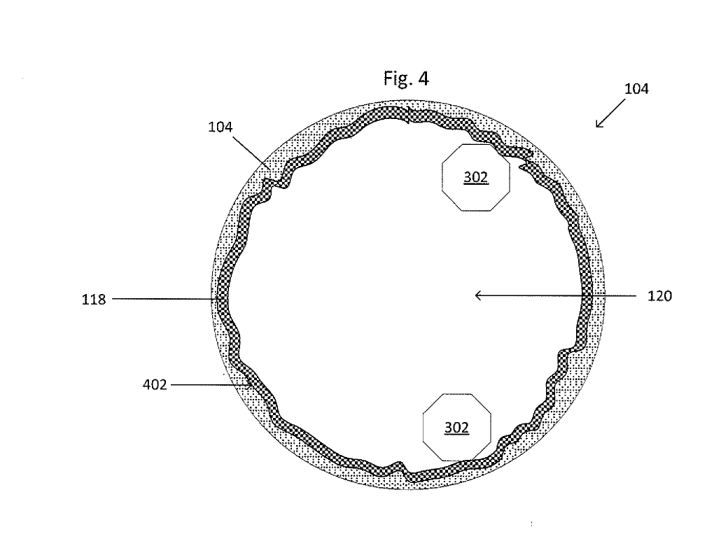

FIG. 4 shows a cross-section of the conduit 104 take along the line I-I. The conduit 104 includes a rough inner surface 402 to create turbulent flow within the conduit 104. The rough inner surface 402 creates eddy currents which can add resistance to the flow of the blood. These eddy currents can create friction between the fluid layers, thereby causing turbulent flow.

### Using a Conduit and Venipuncture System

A needle is first inserted into a blood vessel or port of a patient or test subject. Blood, drawn from the blood vessel or port, passes from the needle to the conduit connected to the needle. The blood then passes through an adapter and into a vessel, such as a tube, which has been punctured or pierced by the adapter.

When the conduit is continuous, thereby extending from the needle directly to the adapter, the conduit may include protrusions or a rough inner surface, and the conduit also includes a coating of a capture molecule, such as EpCAM antibody for instance. As the blood enters the conduit, turbulent flow occurs, thereby causing each portion of the blood to come into contact with the EpCAM antibody coating. When the target analyte, such as a circulating tumor cell (“CTC”) for instance, has an EpCAM biomarker, the CTC can be held to the inner surface of the conduit. The remainder of the blood, such as plasma, red blood cells, and white blood cells for instance, which do not have an EpCAM biomarker, can continue down the conduit, eventually being collected in the vessel.

When the conduit is segmented, such that a first conduit segment extends from the needle to a capture chip and a second conduit segment extends from the capture chip to an adapter, the conduit can be smooth and the capture chip can include at least one protrusion or a rough inner surface. The capture chip also includes a coating of a capture molecule, such as EpCAM antibody for instance. As the blood enters the capture chip, turbulent flow occurs, thereby causing each portion of the blood to come into contact with the EpCAM antibody coating. When the target analyte, such as a CTC for instance, has an EpCAM biomarker, the CTC can be held to the inner surface of the conduit. The remainder of the blood, such as plasma, red blood cells, and white blood cells for instance, which do not have an EpCAM biomarker will exit the capture chip, travel through the second conduit segment, and eventually be collected in the vessel.

After the appropriate amount of blood has been processed, the needle can be removed from the patient's blood vessel or port. A wash may then be flowed through the conduit (and capture chip when present) to remove the unwanted, non-target analytes. To wash, the needle may be removed from the conduit at the respective connectors and a syringe, filled with a wash, may be connected to the conduit. The wash is pushed through. The target analytes can then be fixed, permeabilized, labeled, and/or removed from the conduit. To fix, a fixative (such as formaldehyde, formalin, methanol, acetone, paraformaldehyde, or glutaraldehyde) is flowed through the conduit in a manner similar to that of the wash. To permeabilize, a permeabilizing agent or detergent (such as saponin, polyoxyethylene, digitonin, octyl β-glucoside, octyl β-thioglucoside, 1-S-octyl-β-D-thioglucopyranoside, polysorbate-20, CHAPS, CHAPSO, (1,1,3,3-Tetramethylbutyl)phenyl-polyethylene glycol or octylphenol ethylene oxide) is flowed through the conduit in a manner similar to that of the wash. To label, a labeling agent (such as fluorescently-labeled antibodies, Pap stain, Giemsa stain, or hematoxylin and eosin stains) is flowed through the conduit in a manner similar to that of the wash. To remove the target analyte, proteolytic cleavage, pH variation, or salt concentration variation (i.e. increasing the salt concentration of the surrounding solution to disrupt the molecular interactions that hold the target analyte to the capture molecule) may be performed or flowed through. The target analyte, which may now be severed from the capture molecule, can flow out of the conduit into a collection vessel for isolation and/or farther processing.

Some processing or analysis methods and techniques include, but are not limited to, extracellular analysis and/or intracellular analysis including intracellular protein labeling; nucleic acid analysis, including, but not limited to, DNA microarrays and DNA hybridization arrays; in situ hybridization (“ISH”—a tool for analyzing DNA and/or RNA, such as gene copy number changes); or branched DNA (“bDNA”—a tool for analyzing DNA and/or RNA, such as mRNA expression levels) analysis. These techniques require isolation, permeabilization, and fixation of the target analyte prior to analysis.

The foregoing description, for purposes of explanation, used specific nomenclature to provide a thorough understanding of the disclosure. However, it will be apparent to one skilled in the art that the specific details are not required in order to practice the systems and methods described herein. The foregoing descriptions of specific embodiments are presented by way of examples for purposes of illustration and description. They are not intended to be exhaustive of or to limit this disclosure to the precise forms described. Many modifications and variations are possible in view of the above teachings. The embodiments are shown and described in order to best explain the principles of this disclosure and practical applications, to thereby enable others skilled in the art to best utilize this disclosure and various embodiments with various modifications as are suited to the particular use contemplated. It is intended that the scope of this disclosure be defined by the following claims and their equivalents:

I/We claim:

1. A kit for separating a target analyte from a sample, comprising:
a conduit including a first end and a second end;
a coating with a high affinity for the target analyte deposited on an inner surface of the conduit,
a first needle to connect to the first end of the conduit to permit continuous fluid flow of the sample.

2. The kit of claim 1, wherein the inner surface further comprises at least one protrusion.

3. The kit of claim 1, wherein the inner surface further comprises a rough surface to effect turbulent flow of the sample within the conduit.

4. The kit of claim 1, wherein the coating comprises a capture molecule to capture the target analyte

5. The kit of claim 4, wherein the capture molecule is a primary antibody that binds to a biomarker on the target analyte, a chemo-attractant, or an adhesive.

6. The kit of claim 1, further comprising a collection vessel including a cavity for holding a sample and a pressure regulator to reduce a pressure differential between a blood vessel and the cavity, the collection vessel to connect to the second end of the conduit.

7. The kit of claim 6, wherein the pressure regulator is a valve or a vacuum pump.

8. The kit of claim 1, further comprising a second needle to connect the second end of the conduit to a collection vessel or a place from where the sample originated.

9. The kit of claim 1, wherein the conduit further comprises a flow control to regulate flow of the sample through the conduit.

10. The kit of claim 1, wherein the conduit further comprises a pressure control to regulate the pressure differential between a collection vessel and the first needle.

11. A kit for separating a target analyte from a sample, comprising:
a first needle;
a capture chip including an inner surface;
a conduit to connect the first needle to the inner surface of the capture chip; and
a coating with a high affinity for the target analyte deposited on at least a portion of the inner surface,
the conduit to permit continuous fluid flow between the first needle and the inner surface of the capture chip.

12. The kit of claim 11, wherein the inner surface further comprises at least chip protrusion.

13. The kit of claim 11, wherein the coating comprises a capture molecule to capture the target analyte

14. The kit of claim 13, wherein the capture molecule is a primary antibody that binds to a biomarker on the target analyte, a chemo-attractant, or an adhesive.

15. The kit of claim 11, the conduit further comprising a first conduit segment and a second conduit segment, the first conduit segment to fluidly connect the first needle to the first end of the capture chip and the second conduit segment to fluidly connect the second end of the capture chip to a second needle.

16. The kit of claim 15, the second needle to be inserted into a collection vessel to collect any remaining sample or into a place from where the sample originated.

17. The kit of claim 11, further comprising a collection vessel including a cavity for holding a sample and a pressure regulator to reduce a pressure differential between a blood vessel and the cavity, the collection vessel to connect to the second end of the conduit.

18. The kit of claim 17, wherein the pressure regulator is a valve or a vacuum pump.

19. The kit of claim 11, wherein the conduit further comprises a flow control to regulate flow of the sample through the conduit.

20. The kit of claim 11, wherein the conduit further comprises a pressure control to regulate the pressure differential between a collection vessel and the first needle.
